(19) United States
(12) Patent Application Publication
(10) Pub. No.: US 2007/0104786 A1
(43) Pub. Date: May 10, 2007

(54) Dosage forms for immediate gastric release of a calcium transport stimulator coupled with delayed gastric release of a bis-phosphonate

(75) Inventor: Moshe Fleshner-Barak, Petach Tikva (IL)

Correspondence Address: KENYON & KENYON LLP, ONE BROADWAY, NEW YORK, NY, 10004, US

(73) Assignee: Teva Pharmaceutical Industries Ltd., Petah Tiqva (IL)

(21) Appl. No.: 11/485,712

(22) Filed: Jul. 12, 2006

Related U.S. Application Data
(63) Continuation of application No. 10/196,766, filed on Jul. 17, 2002.
(60) Provisional application No. 60/305,913, filed on Jul. 17, 2001.

(51) Int. Cl. A61K 31/675; A61K 31/663; A61K 31/59; A61K 9/22

(52) U.S. Cl. 424/468; 514/102; 514/89; 514/167

(57) ABSTRACT

The present invention provides a gastric retention dosage form for immediate or uncontrolled release of a vitamin D derivative that stimulates calcium absorption from the intestine, like calcitriol, alphacalcidol and calcifediol, combined with delayed release of a bis-phosphonate calcium resorption inhibitor such as alendronic acid and its pharmaceutically acceptable salts and hydrates.

### COSS-REFERENCE TO RELATED APPLICATION

This application is a continuation of U.S. Ser. No. 10/196,766, which claims the benefit of provisional application Ser. No. 60/305,913, filed Jul. 17, 2001 which is incorporated herein by reference.

### FIELD OF THE INVENTION

The present invention relates to a gastric retention system for immediate release of a vitamin D derivative that stimulates calcium absorption from the intestine, like calcitriol, combined with delayed release of a bis-phosphonate calcium resorption inhibitor such as alendronic acid and its pharmaceutically acceptable salts and hydrates.

### BACKGROUND OF THE INVENTION

Treatment of osteoporosis, metastatic bone disease, and Paget's disease can benefit from improvements in controlled gastric release and multiple dose delivery technology. Bis-phosphonates such as alendronate, risedronate, etidronate and tiludronate are commonly prescribed drugs for treatment of these diseases. Despite their benefits, bis-phosphonates suffer from very poor oral bioavailability. Alendronate has less than 1% bioavailability. Gert, B. J.; Holland, S.D.; Kline, W.F.; Matuszewski, B. K.; Freeman, A.; Quan, H.; Lasseter, K. C.; Mucklow, J. C.; Porras, A. G. “Studies of The Oral Bioavailablity of Alendronate,” Clinical Pharmacology & Therapeutics 1995, 58, 288-298. Its absorption is inhibited by foods and beverages other than water. Id. Side effects experienced by patients who have taken alendronate include irritation of the upper gastrointestinal mucosa. Liberman, U. A.; Hirsch, L. J.; “Esophagitis and Alendronate” N. Engl. J Med., 1996, 335, 1069-70. This irritation can lead to more serious conditions. Physicians' Desk Reference, Fosamax, Warnings.

Alendronate is best absorbed from the upper GI tract (duodenum and jejunum). Lin, J. H. “Bisphosphonates: A Review of Their Pharmacokinetic Properties,” Bone, 1996, 18, 75-85; Porras, A. G.; Holland, S. D.; Gertz, B. J.; “Pharmacokinetics of Alendronate,” Clin. Pharmacokinet 1999, 36, 315-328. Alendronate and is best absorbed at a pH of ˜6. Gert, B. J.; Holland, S. D.; Kline, W. F.; Matuszewski, B. K.; Freeman, A.; Quan, H.; Lasseter, K. C.; Mucklow, J. C.; Porras, A. G. “Studies of The Oral Bioavailablity of Alendronate,” Clinical Pharmacology & Therapeutics, 1995, 58, 288-298. As discussed in commonly-assigned, co-pending application Ser. No. 09/770,898, controlled gastric release of alendronate would allow for extended delivery of the drug to the duodenum and jejunum parts of the intestine and should result in improved bioavailability, and thus allow lower dosing and less irritation.

In addition to bis-phosphonate therapy, options in the treatment of osteoporosis include hormone replacement therapy and calcium supplementation therapy. Kleerekoper, M., Schein, J. R. “Comparative Safety of Bone Remodeling Agents with A Focus on Osteoporosis Therapies,” J. Clin. Pharmacol. 2001, 41, 239. Increased calcium levels can potentially improve the state of bone mineralization in patients with osteoporosis. Over the last thirty years, calcium supplementation, along with vitamin D or vitamin D derivatives such as calcitriol, has been one of the options for treating the problems of osteoporosis. Cannigia, A., Vattimo, A. “Effects of 1,25 Dihydroxycholecalciferol on Calcium Absorption in Postmenopausal Osteoporosis,” Clin. Endocrinol., 1979, 11, 99; Riggs, B. L., Nelson, K. L. “Effect of Long Term Treatment with Calcitriol on Calcium Absorption and Mineral Metabolism in Postmenopausal. Osteoporosis, J. Clin. Endocrinol. Metab. 1985, 61, 457; Reid, I. R., Ames, R. W., Evans, M. C., Gamble, G. D., Sharpe, S. J. “Long Term Effects of Calcium Supplementation on Bone Loss and Fracture in Post-menopausal Women, a Randomized Controlled Trial, Am. J Med., 1995, 98, 331. Calcitriol (1,25-dihydroxyvitamin D₃) is a vitamin D derivative that is active in the regulation of the absorption of calcium from the gastrointestinal tract. Physicians' Desk Reference, Rocaltrol Oral Solution, Description. Calcitriol is the biologically active form of vitamin D₃ and stimulates intestinal calcium transport. Merck Index, 12th Ed., 1681. Calcitriol is rapidly absorbed from the intestine and reaches peak serum concentrations within three to six hours after ingestion. Physicians' Desk Reference, Rocaltrol Oral Solution, Pharmacokinetics. Calcitriol is used to treat calcium deficiency.

Over the past several years, successful trials have been performed that confirm that there is a synergistic effect in using a combined therapy of calcitriol and bis-phosphonates. Frediani, B., Allegri, A., Bisogno, S., Marcolongo, R. “Effects of Combined Treatment with Calcitriol Plus Alendronate on Bone Mass and Bone Turnover in Postmenopausal Osteoporosis-Two Years of Continuous Treatment,” Clin. Drug Invest. 1998, 15, 223; Masud, T., Mulcaby, B., Thompson, A. V., Donnolly, S., Keen, R. W., Doyle, D. V., Spector, T. D., “Effects of Cyclical Etidronate Combined with Calcitriol Versus Cyclical Etidronate Alone on Spine and Femoral Neck Bone Mineral Density in Postmenopausal Women,” Ann. Rheum. Dis., 1998, 57, 346; Malvolta, M., Zanardi, M., Veronesi, M., Ripamonti C., Gnudi, S. “Calcitriol and Alendronate Combination Treatment in Menopausal Women with Low Bone Mass,” Int. J Tissue React. 1999, 21, 51; Nuti, R., Martini, G., Giovani, S., Valenti, R. “Effect of Treatment with Calcitriol Combined with Low-dosage Alendronate in Involutional Osteoporosis,” Clin. Drug Invest., 2000, 19, 56. The goal of the combined therapy trials is to improve therapeutic results and lower the dosage of the two drugs. In these trials the drugs were given individually. International Publication WO 2001/028564 discloses a tablet containing a combination of calcitriol and alendronate in a particular range of ratios of the two drugs.

There is a need for an improved dosage regimen for the combination therapy of a vitamin D analog (e.g. calcitriol) and bis-phosphonates (e.g. alendronate).

For reasons that will become apparent from the description of the invention, it would be highly desirable in combination therapy with a bis-phosphonate and a calcium transport stimulator to be able to release the bis-phosphonate in the patient's stomach after the vitamin D derivative has been released. However, the average residence time of a pharmaceutical tablet in the stomach is about an hour. Thus, a pharmaceutical dosage form may pass through the stomach and into the intestine before the active ingredient has been completely released, especially if the dosage form delays or sustains the release of the active ingredient. If the dosage form is retained in the stomach, however, the bis-phosphonate could be released an hour or more after the vitamin D derivative upstream of the small intestine where the bis-phosphonate is most readily absorbed.

Formulation specialists have developed methods to increase the retention time of oral dosage forms in the stomach. One of the general methods involves using an intragastric expanding dosage form that swells upon contact with stomach juices, preventing its passage through the pylorus. Some intragastric expanding dosage forms use hydrogels, which expand upon contact with water, to expand the dosage form to sufficient size to prevent its passage through the pylorus. An example of such a dosage form is described in U.S. Pat. No. 4,434,153.

As reviewed by Hwang, S. et al. “Gastric Retentive Drug-Delivery Systems,” Critical Reviews in Therapeutic Drug Carrier Systems, 1998, 15, 243-284, one of the major problems with intragastric expanding hydrogels is that it can take several hours for the hydrogel to become fully hydrated and to swell to sufficient size to obstruct passage through the pylorus. Since food remains in the stomach on average from about 1 to 3 hours, there is a high probability that known expanding dosage forms like that of the '153 patent will pass through the pylorus before attaining a sufficient size to obstruct passage. The rate-limiting factor in the expansion of ordinary hydrogels is the rate of delivery of water to non-surficial hydrogel material in the dosage form. Conventional non-hydrated hydrogels are not very porous when dry and ingress of water into the hydrogel is slowed further by the formation of a low permeability gelatinous layer on the surface after initial contact with water. Thus, there remains a need in the art for improved gastric retention systems that expand rapidly to retain a controlled gastric release dosage form in the patient's stomach.

In combination with developing improved controlled release systems, those in the art are developing delivery systems that deliver multiple doses of a medication by administration of a single dose unit. An example of such a delivery system is described in U.S. Pat. No. 5,837,248. The '248 patent discloses an improved dosing of a medication whereby two or more effective, time-separated doses may be provided by administration of a single dose unit comprising two groups of particles: immediate-release particles and delayed-release particles, both containing the same active drug.

Although there has been a recognition of the benefits of combination therapy in the treatment of osteoporosis, metastatic bone disease and Paget's disease, and although there have been advances in controlled release systems for multi-dose medications, there remains a need for an improved controlled delivery system for a bis-phosphonate and a calcium transport stimulator in order to fully realize the advantages of combined therapy.

### SUMMARY OF THE INVENTION

If a bis-phosphonate calcium resorption inhibitor is delivered to the upper small intestine after delivery of a vitamin D derivative capable of stimulating transport of calcium from the intestine to the bloodstream, absorption of the bis-phosphonate will be improved. The bis-phosphonate will enter an environment partially depleted in calcium due to the transport activity of the vitamin D derivative. This depleted calcium environment will thus allow a higher absorption of the bis-phosphonate, thereby allowing a dose lowering in addition to the dose lowering caused by the synergistic effect of the bis-phosphonate and vitamin D derivatives that occurs after reaching the bloodstream. It takes an hour or more after entering the intestine for the vitamin D derivative calcitriol to attain maximum activity. Thus, the bis-phosphonate must be retained in the stomach for at least that long so that it may be released at an optimum time for maximum absorption.

Commonly-assigned co-pending U.S. patent applications Ser. Nos. 09/770,898 and 09/887,204, which are hereby incorporated by reference in their entirety, describe rapidly expanding compositions and oral dosage forms that swell rapidly in the gastric juices of a patient, thereby increasing the likelihood that active ingredient(s) carried by the dosage form will be released in the stomach.

The present invention provides rapidly expanding oral dosage forms that contain a bis-phosphonate, a vitamin D derivative and the rapidly expanding composition. The dosage forms release the vitamin D derivative immediately upon entering the stomach, while the release of bis-phosphonate into the stomach is delayed until the vitamin D derivative has depleted calcium from the upper small intestine.

In another aspect, the present invention relates to a method for providing a combination drug regimen combining separate pre-dosing with a vitamin D derivative followed by dosing with a bis-phosphonate.

### BRIEF DESCRIPTION OF THE DRAWINGS

FIG. 1 depicts the concentration of alendronate in dog urine as a function of time post alendronate dosing. Session 1, alendronate only; session 2, calcitriol pre-dose followed 2 hours later by alendronate.

### DETAILED DESCRIPTION OF THE INVENTION

An object of the invention is to provide dosage forms that enable improvement in combination therapy with bis-phosphonates and calcium transport stimulators like calcitriol. The improved therapy is realized with this invention by taking advantage of the fact that a calcium transport stimulator depletes the calcium concentration in the intestine, in addition to its recognized benefit of increasing calcium in the blood. Complexation of a bis-phosphonate with calcium in the gut inhibits its absorption. Thus, there is a previously unrecognized potential benefit of increasing the bioavailability of the bis-phosphonate through combined therapy. However, there is a delay of several hours between when the calcitriol enters the intestine and when the blood calcium level peaks. Maximum calcium depletion in the intestine should coincide with the peak in blood calcium level. Therefore, in order to release the bis-phosphonate into an environment maximally depleted of calcium, the bis-phosphonate must be retained in the stomach and its release must be delayed for several hours.

Thus, in one embodiment, the present invention provides a method of improving the bioavailability of a bis-phosphonate, especially, alendronate, by administering a combination drug regimen that includes the steps of administering a pre-dose of a vitamin D derivative and, about 2 to about 6 hours later, administering a therapeutic dose of a bis-phosphonate. The vitamin D derivatives and bis-phosphonates useful in the practice of this and other embodiments herein described are the same. Preferably, the vitamin D derivative is calcitriol and the bis-phosphonate is alendronate.

Administration of the vitamin D analog in the combination drug regimen can be by any means known in the art. Solid oral dosage forms are preferred.

Administration of the bis-phosphonate in the combination drug regimen can also be by any means known in the art. Administration via a solid oral dosage form is preferred. The solid oral dosage form can be of the conventional type well known in the art (e.g. Fosamax®), or it can be of the gastric retention type herein described.

The therapeutic or prophylactic doses of vitamin D derivative and bis-phosphonate to be administered in this combination drug regimen are the same as in other embodiments of the invention.

The dosage forms of another embodiment of the present invention enable improved combination therapy with bis-phosphonates and calcium transport stimulators by releasing the calcium transport stimulator in an immediate or uncontrolled manner, by swelling to a size that prevents passage through the pylorus and by releasing the bis-phosphonate in the stomach after a delay time period to allow the calcium transport stimulator to deplete the upper GI tract of calcium. After a delay period of preferably an hour or more, more preferably from about 2 to about 6 hours, the bis-phosphonate is released in the stomach in either an immediate or sustained release manner. Afterwards, the swollen tablet degrades or erodes into particles that are sufficiently small to traverse the pylorus.

Preferably, the pharmaceutical dosage form is retained in the stomach for about three hours or more before it breaks up, more preferably about five hours or more. In order to obstruct passage through the pylorus, the dosage form preferably swells by a factor of three or more, more preferably about eight or more, within about fifteen minutes of contacting gastric fluid. Yet more preferably, such swelling is reached within about five minutes.

Bis-phosphonates useful as calcium resorption inhibitors in the present invention include, for example, alendronate, risedronate, etidronate and tiludronate. The most preferred bis-phosphonate is alendronate. It will be understood that the bis-phosphonate can be in the form of a pharmaceutically acceptable salt, a hydrate, or the hydrate of a pharmaceutically acceptable salt.

The dosage level of the bis-phosphonate will depend in part upon whether the dosage form is intended for delayed release or delayed/sustained release of the bis-phosphonate. A non-sustained release alendronate formulation preferably contains from about 2 mg to about 40 mg of alendronate. A delayed/sustained release alendronate formulation preferably contains from about 6 to about 120 mg of alendronate. A non-sustained release risedronate formulation preferably contains from about 20 to about 40 mg of risedronate. A delayed/sustained release risedronate formulation preferably contains from about 60 to about 120 mg of risedronate. A non-sustained release etidronate formulation preferably contains from about 200 mg to about 400 mg of etidronate. A delayed/sustained release etidronate formulation preferably contains from about 600 to about 1200 mg of etidronate. A non-sustained release tiludronate formulation preferably contains from about 200 mg to about 300 mg of tiludronate. A delayed/sustained release tiludronate formulation preferably contains from about 600 mg to about 1200 mg of tiludronate.

The bis-phosphonate may be provided in any pharmaceutically acceptable salt or acid form, salts being generally preferred because they cause less membrane irritation. Alendronate is preferably provided as a monosodium salt monohydrate or trihydrate. Risedronate is preferably provided as a monosodium salt hemipentahydrate. Etidronate and tiludronate are preferably provided as hydrated or anhydrous disodium salts.

Vitamin D derivatives useful as calcium transport stimulators include calcitriol, alphacalcidol, 24,25-dihydroxy vitamin D₃, and calcifediol. The most preferred calcium transport stimulator of the present invention is calcitriol. The calcium transport stimulator may be dosed in any amount that results in increased intestinal absorption of the bis-phosphonate compared to an equal dose of the bis-phosphonate administered without the calcium transport stimulator. A preferred dosage range is from about 0.01 μg to about 0.5 μg. A most preferred dosage is about 0.05 μg.

The dosage forms of this invention are retained in the stomach for an extended period of time by swelling rapidly on contact with aqueous solution, such as gastric fluid. The term “gastric fluid” means the endogenous fluid medium of the stomach, including water and secretions, or simulated gastric fluid. “Simulated gastric fluid” means any fluid that is generally recognized as providing a useful substitute for authentic gastric fluid in experiments designed to assess the chemical or biochemical behavior of substances in the stomach. One such simulated gastric fluid is USP Gastric Fluid TS, without enzymes. United States Pharmacopeia and National Formulary 24/19 p. 2235 (1999). Thus, it will be understood that throughout this disclosure and in the claims “gastric fluid” means authentic gastric fluid or simulated gastric fluid.

Rapid swelling is achieved by a gastric retention composition. The gastric retention composition may comprise a combination of a hydrogel, a superdisintegrant, and tannic acid. This composition is further described in our commonly assigned co-pending U.S. patent applications Ser. Nos. 09/770,898 and 09/887204, previously incorporated by reference in their entirety.

The preferred hydrogel of the gastric retention composition is hydroxypropyl methylcellulose, either alone or in combination with hydroxypropyl cellulose and/or a cross-linked acrylate polymer. Suitable cross-linked acrylate polymers include polyacrylic acid cross-linked with allyl sucrose and polyacrylic acid cross-linked with divinyl glycol. As further illustrated in the Examples, a preferred hydrogel of the invention is a mixture of hydroxypropyl methylcellulose and hydroxypropyl cellulose. The most preferred hydrogel of the present invention is a combination of hydroxypropyl methylcellulose and hydroxypropyl cellulose in a weight ratio of from about 1:3 to about 5:3. The molecular weight of the hydrogels is not critical to practice of the invention.

The gastric retention composition also may include a superdisintegrant. Superdisintegrants are pharmaceutical excipients within a larger class of excipients known as disintegrants. Disintegrants are typically hydrophilic polymers of either natural or synthetic origin. Superdisintegrants are disintegrants that swell upon contact with water. Preferred superdisintegrants of the present invention swell to at least double their non-hydrated volume on contact with water. Exemplary of these superdisintegrants are cross-linked polyvinyl pyrollidone (a.k.a. crospovidone), cross-linked carboxymethyl cellulose sodium (a.k.a. croscarmellose sodium) and sodium starch glycolate. The most preferred superdisintegrant is croscarmellose sodium.

The gastric retention composition further may include tannic acid. Tannic acid, also called tannin, gallotannin and gallotannic acid, is a naturally occurring constituent of the bark and fruit of many trees. The term “tannins” conventionally refers to two groups of compounds, “condensed tannins” and “hydrolyzable tannins.” Merck Index monograph No. 8828 (9th ed. 1976). The hydrolyzable tannins are sugars that are esterified with one or more (polyhydroxylarene) formic acids. One common polyhydroxylarene formic acid is galloyl (i.e. 3,4,5-trihydroxybenzoyl). Another common polyhydroxylarene formic acid substituent of tannins is meta-digallic acid. A common sugar moiety of tannins is glucose. The tannic acid of the present invention is selected from the hydrolyzable tannins, and especially glucose tannins in which one or more of the hydroxyl groups of glucose is esterified with gallic acid and/or meta-digallic acid. USP tannic acid is preferred for use with this invention.

The preferred gastric retention composition comprises a hydrogel, a superdisintegrant and tannic acid. These excipients more preferably are combined in a weight ratio, exclusive of the active ingredients and any other excipients that may be present, of from about 20 wt. % to about 80 wt. % hydrogel, from about 10 wt. % to about 75 wt. % superdisintegrant and from about 2 wt. % to about 15 wt. % tannic acid. A yet more preferred composition comprises from about 10 wt. % to about 35 wt. % superdisintegrant, about 5 wt. % (±2 wt. %) tannic acid, plus an amount of hydrogel sufficient to bring the total to 100 wt. %.

One especially preferred gastric retention composition comprises from about 10 wt. % to about 20 wt. % hydroxypropyl methyl cellulose, from about 45 wt. % to about 50 wt. % hydroxypropyl cellulose, from about 25 wt. % to about 35 wt. % sodium starch glycolate and from about 4 wt. % to about 6 wt. % tannic acid.

A second especially preferred gastric retention composition comprises from about 10 wt. % to about 30 wt. % hydroxypropyl methyl cellulose, from about 40 wt. % to about 60 wt. % hydroxypropyl cellulose, from about 7 wt. % to about 35 wt. % sodium croscarmellose and from about 4 wt. % to about 12 wt. % tannic acid.

The dosage form may be prepared conventionally by dry blending, dry granulation or wet granulation of the active ingredients and the gastric retention composition and any other desired excipients.

In a dry granulation, the active ingredients and excipients may be compacted into a slug or a sheet and then comminuted into compacted granules. The compacted granules may be compressed subsequently into a final dosage form. It will be. appreciated that the processes of slugging or roller compaction, followed by comminution and recompression render the hydrogel, superdisintegrant, tannic acid, and active ingredients intragranular in the final dosage form. Alternatively, any of the active ingredients or excipients of the gastric retention composition may be added after comminution of the compacted composition, which results in that active ingredient or excipient being extragranular.

As an alternative to dry granulation, the blended composition may be compressed directly into the final pharmaceutical dosage form using direct compression techniques. Direct compression produces a more uniform tablet without granules. Thus, the active ingredients and any other desired excipients are blended with the composition prior to direct compression tableting. Such additional excipients that are particularly well suited to direct compression tableting include microcrystalline cellulose, spray dried lactose, dicalcium phosphate dihydrate, and colloidal silica.

An additional alternative to dry granulation is wet granulation. The blend of excipients may be granulated using an alcohol or water and alcohol mixture as a granulation solvent by standard granulation techniques known in the art followed by drying, sieving, milling and compressing into the final dosage form.

The active ingredients and gastric retention composition may be compacted using conventional compression techniques.

In a preferred dosage form of the invention, a core containing the bis-phosphonate is embedded in the gastric retention composition. Embedded tablets are an example of an embedded core type of dosage form. The dosage form may be formulated to contain the vitamin D derivative in the gastric retention composition or in a coating that is soluble in gastric fluid. A coating of the vitamin D derivative is applied over the gastric retention composition. In either formulation, the vitamin D derivative is released immediately in the stomach and will find its way to the intestine quite rapidly.

The following is an example of an immediate release bis-phosphonate core that may be used to prepare a bis-phosphonate/calcium transport stimulator dosage form of this invention. An immediate release core of bis-phosphonate may be prepared by blending the bis-phosphonate with microcrystalline cellulose, lactose, magnesium stearate and, optionally, a superdisintegrant, and compressing the blend. An exemplary formulation contains from about 20 to about 50 wt. % microcrystalline cellulose, from about 50 to about 80 wt. % lactose, from about 0.5 to about 2 wt. % magnesium stearate and from about 0 to about 5 wt. % crospovidone, sodium croscarmellose or sodium starch glycolate, plus the intended dosage of bis-phosphonate.

The following is an example of a sustained release bis-phosphonate core that may be used to prepare a bis-phosphonate/calcium transport stimulator dosage form of this invention. A sustained release core of bis-phosphonate may be prepared by blending the bis-phosphonate with hydroxypropyl methylcellulose, lactose and magnesium stearate. An exemplary formulation contains from about 5 to about 80 wt. % hydroxypropyl methylcellulose, from about 20 to about 95 wt. % lactose and from about 0.5 to about 2 wt. % magnesium stearate, plus the intended dose of bis-phosphonate.

The core may also be coated with a delayed release coating. Suitable coating substances for forming a delayed release coating include arabinogalactan; carboxymethylcellulose; gelatin; gum arabic; hydroxyethylcellulose; methylcellulose; polyvinyl alcohol; water insoluble resins such as ethyl cellulose, e.g., Ethocel™, polyamide, polymethacrylate, e.g., Eudragit™ NE, Eudragit™ RS, Eudragit™ RL, and silicones; waxes and lipids such as paraffin, carnauba wax, spermaceti, beeswax, stearic acid, stearyl alcohol and glyceryl stearates; and enteric resins such as cellulose acetate phthalate, polyvinyl acetate, hydroxypropyl methylcellulose acetate, Eudragit™ L and Eudragit™ S. The glyceryl esters may be mixed with a wax as previously described in U.S. Pat. No. 4,764,380, which is incorporated by reference in its entirety. Additional coating materials that may be used are disclosed in U.S. Pat. Nos. 4,434,153; 4,721,613; 4,853,229; 2,996,431; 3,139,383 and 4,752,470, which are hereby incorporated by reference in their entirety.

The core also may be coated with a sustained release coating to further slow release of the bis-phosphonate. Such coating materials include polymethacrylate, e.g., Eudragit™ NE, Eudragit™ RS, Eudragit™ RL, Eudragit™ L, Eudragit™ S, and mixtures of hydrophilic and hydrophobic film forming agents. Hydrophilic film forms include methyl cellulose, hydroxypropyl methylcellulose, cellulose phthalate, cellulose acetate phthalate and polyvinyl alcohol. Hydrophobic film forming agents include ethyl cellulose, cellulose acetate, hydroxypropyl methylcellulose phthalate, polyvinyl alcohol maleic anhydride copolymers, β-pinene polymers rosin, partially hydrogenated rosin and glycerol esters of rosin. A sustained release coating may be applied by methods known in the art such as by fluid bed or pan coating techniques.

The core may be embedded in the gastric composition using commercially available equipment such as a Kilian RUD-20 press coat machine.

The vitamin D derivative may be dispersed in the shell of the gastric retention composition. Thus, the vitamin D derivative may be incorporated into the preferred embedded core type dosage form by simply blending with the gastric retention composition before compression in the press coat machine.

The vitamin D derivative may be applied in a coating over the shell. The vitamin D derivative may, for example, be dissolved in ethanol with 0.1 wt. % to about 10 wt. % hydroxypropyl cellulose and then pan coated or spray coated onto the shell using coating techniques that are well known in the art.

Another preferred dosage form embodiment is a capsule. The capsule encapsulates two tablets. One tablet contains the above-described core containing the bis-phosphonate embedded in a shell of the gastric retention composition. The other tablet may be any conventional immediate release formulation containing the vitamin D derivative.

In addition to the above-described excipients, the bis-phosphonate/calcium transport stimulator dosage form may further include one or more other excipients added for any of a variety of other purposes. It will be understood by those in the art that some substances serve more than one purpose in a dosage form. For instance, some substances are binders that help hold a tablet together after compression, yet are disintegrants that help break the tablet apart once it reaches a patient's stomach. It will be further understood that the hydrogel, superdisintegrant and tannic acid of the expanding composition may serve to perform additional functions in the dosage form, which functions may already be known to those skilled in the art.

Further increase in retention times may be realized by adding a compound that produces gas when contacted with acid, such as sodium bicarbonate. Sodium bicarbonate may be provided by blending into the gastric retention composition. Sodium bicarbonate is preferably used at low concentration, of from about 0.5 wt % to about 5 wt. % of expanding composition.

Diluents increase the bulk of a solid pharmaceutical product and may make it easier for the patient and care giver to handle. Diluents include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol and talc.

Compacted dosage forms like those of the present invention may include excipients whose functions include helping to bind the active ingredient and other excipients together after compression. Binders for solid pharmaceutical compositions include, but are not limited to, acacia, alginic acid, carbomer (e.g., carbopol), carboxymethylcellulose sodium, dextrin, ethyl cellulose, gelatin, glucose, guar gum, hydrogenated vegetable oil, hydroxyethyl cellulose, hydroxypropyl cellulose (e.g., Klucel®), hydroxypropyl methylcellulose (e.g., Methocel®), liquid glucose, magnesium aluminum silicate, maltodextrin, methylcellulose, polymethacrylates, polyvinylpyrrolidone (e.g., Kollidon®, Plasdone®), starch, pregelatinized starch, sodium alginate and alginate derivatives.

The dissolution rate of a compacted dosage form in the patient's stomach also may be adjusted by the addition of a disintegrant or second superdistegrant to the dosage form, in addition to the superdisintegrant of the present inventive composition. Such additional disintegrants include, but are not limited to, alginic acid, carboxymethylcellulose calcium, carboxymethylcellulose sodium, colloidal silicon dioxide, croscarmellose sodium (e.g., Ac-Di-Sol®, Primellose®), crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®) and starch.

Glidants can be added to improve the flow properties of a solid composition and improve the accuracy of dosing. Excipients that may function as glidants include, but are not limited to, colloidal silicon dioxide, magnesium trisilicate, powdered cellulose, starch, talc and tribasic calcium phosphate.

When a dosage form such as a tablet is made by compaction, a composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product from the dye. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, surfactants, talc, waxes and zinc stearate.

Flavoring agents and flavor enhancers make the dosage form more palatable to the patient. Common flavoring agents and flavor enhancers for pharmaceutical products that may be included in the dosage forms of the present invention include, but are not limited to, maltol, vanillin, ethyl vanillin, menthol, citric acid, fumaric acid ethyl maltol, and tartaric acid.

The dosage forms may also be colored using any pharmaceutically acceptable colorant to improve their appearance and/or facilitate patient identification of the product and unit dosage level.

Having thus described the invention with reference to certain preferred embodiments, it is further illustrated by the following non-limiting examples.

### EXAMPLES

### Example 1

Sodium alendronate monohydrate is formulated into an extended release core of 5-mm diameter with a composition shown in Table 1 by mixing the powders and direct compression in a standard rotary tablet press. Tablet hardness is between 7 and 12 kP.

TABLE 1

| Component | Weight (mg) |
|---|---|
| Sodium alendronate monohydrate | 11.6 mg* |
| Hydroxypropyl methylcellulose | 25 mg |
| Lactose | 25 mg |
| Magnesium stearate | 0.5 mg |

*equivalent to 10 mg alendronic acid

Calcitriol, 0.05 mg, is dissolved in 20 ml of ethanol. HPMC, 136 g, is granulated with the ethanol solution for two minutes in a high shear mixer (e.g. Diosna). The granulate is dried at 40° C. and milled through a 0.63 mm sieve. The calcitriol granulate is then dry mixed with 400 g of HPC, 80 g of tannic acid and 176 g croscarmellose sodium for five minutes. Magnesium stearate, 8 g, is then added and the mixture is mixed for another minute. The proportions of the blend are given in Table 2. The core is embedded in 800 mg of the blend by compression in a Kilian RUD-20 press coat machine. The outer tablet is of oval shape with dimensions about 17×7×9 mm.

TABLE 2

| Component | weight % |
|---|---|
| Calcitriol* | 6.25 × 10⁻⁶ |
| HPMC (Methocel K-15M) | 17 |
| Tannic acid | 10 |
| HPC (Klucel HF) | 50 |
| Crosscarmelose (aci-di-sol) | 22 |
| Magnesium stearate | 1 |

*Calcitriol is dosed at 0.05 μg per tablet

The resulting tablet provides immediate gastric release of calcitriol and delayed gastric release of alendronate after 2 h. Alendronate is released over about 4 h.

### Example 2

Sodium alendronate monohydrate is formulated into an immediate release core of 5-mm diameter with the composition of Table 3 by mixing the powders and direct compression in a standard rotary tablet press. Tablet hardness is between 7 and 12 kP.

TABLE 3

| Component | Weight (mg) |
|---|---|
| Sodium alendronate monohydrate | 11.6 mg* |
| Microcrystalline cellulose | 30 mg |
| Lactose for direct compression | 20 mg |
| Magnesium stearate | 0.5 mg |

*equivalent to 10 mg alendronic acid

Calcitriol is granulated and the gastric retention blend is prepared as described in Example 1. The core is embedded in 800 mg of the blend by compression in a Kilian RUD-20 press coat machine. The outer tablet is of oval shape with dimensions about 17×7×9 mm. The resulting tablet provides immediate gastric release of calcitriol and delayed gastric release of alendronate that begins after about 2 h. Alendronate is released over about 1 h.

### Example 3

A core containing monosodium alendronate monohydrate is prepared as described in Example 1. The core is embedded into 800 mg of the gastric retention composition of Table 4 formed by dry mixing of the components and compression in a Kilian RUD-20 press coat machine. The outer tablet is of oval shape with dimensions approximately 17×7×9 mm.

TABLE 4

| GRDS Component | weight % |
|---|---|
| HPMC (Methocel ® K-15M) | 17 |
| Tannic acid | 10 |
| HPC (Klucel ® HF) | 50 |
| Crosscarmelose (aci-di-sol ®) | 22 |
| Magnesium stearate | 1 |

Eight hundred grams of these tablets are coated by dissolving 25 g of HPC LF in 2 L of ethanol. Calcitriol, 0.05 mg, is dissolved in 20 ml of ethanol and added to the HPC solution. The solution is mixed for one minute. The tablets are spray coated in a perforated pan coater at a bed temperature of about 35° C. and air inlet temperature of 45 ° C. The tablets are air dried until the bed temperature reaches 45 ° C. The resulting tablets have a uniform coating containing 0.05 μg of calcitriol per tablet.

### Example 4

### In-Vivo Study of the Effect of Delivering Alendronate as a Combination Drug Regimen with Calcitriol

An in vivo study in an animal model was conducted to determine whether the novel combination drug regimen of calcitriol and alendronate improves the bioavailability of alendronate.

Six female beagle dogs, each approximately 2 years old and weighing approximately 9 kg were the animal models in this study. The same animals were used in each of two separate treatment sessions lasting 22-24 hours each. There was a 7 day wash-out period between sessions. The clinical state of each dog was checked within 48 hours prior to each treatment session and again after the last session. In each session the animals were dosed in the fasted state (n.p.o. 10-12 hours). The dogs were fed a standardized meal (200-250 g, Shur-Gain, Canada) four hours after dosing with alendronate.

During each session, the dogs were housed in steel metabolic cages. Urine samples were recovered from the bottom of the metabolic cages. At each collection point, a representative sample of urine (ca. 15 ml) was taken in a capped polypropylene vial and immediately frozen at −20° C. The remainder of the sample was frozen and retained.

Urine samples were analyzed for alendronate by HPLC (Anapharm, Canada).

In each session, the study drug was administered in the AM, in the fasted state, with 250 ml water (regulated at pH=2) administered via gastroesophogeal tube. During the monitoring (collection) period of each session, dogs were hydrated orally (syringe) every two hours with 200-250 ml water. As noted above, a meal was allowed 4 hours after the administration of alendronate.

In the first (reference) study session, alendronate (10 mg, Fosamax®) was administered in 250 ml pH regulated water via a gastroesophegeal tube.

In the second study session, a pre-dose of calcitriol (0.25 μg, Rocaltrol®) was administered with 10-20 ml tap water. Two hours following the clacitriol pre-dose, alendronate (10 mg, Fosamax) was administered with 250 ml pH regulated water via gastroesophogeal tube.

Cumulative levels of alendronate in urine was determined at 0, 3, 6, 9, and 12 hours following alendronate dosage.

The results of the analyses of alendronate in urine for the two treatments are reported in Tables 1 and 2 and the average values are compared graphically in FIG. 1. In five of six dogs, the Fosamax® (Table 1) gave a total of ˜225-300 μg of alendronate in the urine over 12 hours. The sixth dog gave very low values but there were analytical problems with two of the urine samples, including the three hour sample which should have the highest values. The average value, without the data for dog #648, is 257.6 μg, and with all the data 225.9 μg, for a bioavalability of 2.6% or 2.3% respectively. These values are close to literature values in the dog. When the dogs were pretreated with calcitriol, followed by alendronate 2 hours later, the values of alendronate found in the urine were higher. The values ranged from 322 μg to 1016 μg. The average value was 527.5 μg. This value translates into a bioavailability of 5.3 % which is twice as high as the value without the pretreatment. It is noted that the bioavailability was as high as 10% in one of the dogs.

TABLE 1
Alendronate Excreted in Dog Urine - Fosamax ®
Fosamax - fasted

| | | Alendronate (ug) | | | | | |
|---|---|---|---|---|---|---|---|
| | | excreted in Dog Urine | | | | | |
| | time (hr) | 0 | 3 | 6 | 9 | 12 | TOTAL |
| animal # | 295 | blq | 257.51 | 22.92 | 7.34 | 4.55 | 292.32 |
| | 109 | blq | 185.57 | 35.04 | 4.55 | 2.82 | 227.98 |
| | 612 | blq | 188.77 | 42.71 | 15.71 | 4.39 | 251.58 |
| | 648 | blq | nrv | 27.94 | 7.51 | Nrv | 35.45 |
| | 005 | blq | 181.03 | 72.97 | 15.46 | Nrv | 269.46 |
| | 578 | blq | 211.94 | 52.25 | 9.71 | 4.62 | 278.52 |
| | avg = | 0 | 205.0 | 42.3 | 10.0 | 4.1 | 225.9 |
| | avg | | 205.0 | 44.7 | 10.5 | 4.1 | 257.6 |
| | w/o648 | | | | | | |

blq = below level of quantitation
nrv = no reported value

TABLE 2
Alendronate Excreted in Dog Urine - Rocaltro ® + Fosamaxä ®
Rocaltrol + Fosamax- fasted

| | | Alendronate (ug) excreted in Dog Urine | | | | | |
|---|---|---|---|---|---|---|---|
| | time (hr) | 0 | 3 | 6 | 9 | 12 | TOTAL |
| animal # | 295 | blq | 442.05 | 75.91 | 6.13 | 8.05 | 532.14 |
| | 109 | blq | 362.84 | 22.1 | 9.57 | 8.96 | 403.47 |
| | 612 | blq | 280.88 | Nrv | 30.54 | 11.36 | 322.78 |
| | 648 | blq | 941.1 | Nrv | 8.51 | 66.01 | 1015.62 |
| | 005 | blq | 407.82 | 41.15 | 5.94 | 10.12 | 465.03 |
| | 578 | 2.62 | 396.87 | 23.16 | nrv | 6.01 | 426.04 |
| | avg = | 2.6 | 471.9 | 40.6 | 12.1 | 18.4 | 527.5 |

Having thus described the invention with reference to various preferred embodiments, those skilled in the art will appreciate modifications of these exemplary embodiments that do not depart from the spirit and scope of the invention as defined by the claims that follow.

What is claimed is:

1. An oral pharmaceutical dosage form that provides immediate or uncontrolled release of a vitamin D derivative to stimulate intestinal absorption of calcium and delayed release of a therapeutic bis-phosphonate at least about an hour after the vitamin D derivative is released.

2. The pharmaceutical dosage form of claim 1 wherein release of the bis-phosphonate begins at least about two hours but not later than about six hours after the vitamin D derivative is released.

3. The pharmaceutical dosage form of claim 1 wherein the vitamin D derivative is selected from the group consisting of calcitriol, alphacalcidol, 24,25-dihydroxy vitamin D₃, and calcifediol.

4. The pharmaceutical dosage form of claim 3 wherein the vitamin D derivative is calcitriol.

5. The pharmaceutical dosage form of claim 1 wherein the bis-phosphonate is selected from the group consisting of alendronate, risedronate, etidronate and tiludronate.

6. The pharmaceutical dosage form of claim 5 wherein the bis-phosphonate is alendronic acid or a pharmaceutically acceptable salt or hydrate thereof.

7. The pharmaceutical dosage form of claim 6 wherein the bis-phosphonate is monosodium alendronate monohydrate.

8. The pharmaceutical dosage form of claim 6 wherein the bis-phosphonate is monosodium alendronate trihydrate.

9. The pharmaceutical dosage form of claim 1 wherein the pharmaceutical dosage form comprises a hydrogel.

10. The pharmaceutical dosage form of claim 9 wherein the hydrogel comprises hydroxypropyl methylcellulose and hydroxypropyl cellulose in a weight ratio of from about 1:3 to about 5:3.

11. The pharmaceutical dosage form of claim 9 wherein the dosage form further comprises a superdisintegrant.

12. The pharmaceutical dosage form of claim 11 wherein the superdisintegrant is selected from the group consisting of cross-linked polyvinylpyrrolidone, cross-linked carboxymethyl cellulose sodium and sodium starch glycolate.

13. The pharmaceutical dosage form of claim 11 wherein the dosage form further comprises tannic acid.

14. A pharmaceutical dosage form of claim 1 that swells by a factor of three or more within about fifteen minutes of contacting aqueous solution.

15. A pharmaceutical dosage form of claim 14 that swells by a factor of eight or more within about five minutes of contacting aqueous solution.

16. An oral pharmaceutical dosage form for administration to a patient to treat bone disease comprising a compacted core containing a therapeutic bis-phosphonate embedded in a shell comprising a vitamin D derivative that stimulates transport of calcium from the intestine into the bloodstream, wherein the shell expands upon contact with gastric fluid to promote retention of the dosage form in the patient's stomach for a prolonged period of time, the vitamin D derivative is released from the shell, the bis-phosphonate is released at least about an hour after the vitamin D derivative is released, and the dosage form degrades into particles too small too cause gastric retention.

17. The oral pharmaceutical dosage form of claim 16 wherein the core slows release of the bis-phosphonate.

18. The oral pharmaceutical dosage form of claim 16 wherein the core has a coating that slows or delays release of the bis-phosphonate.

19. An oral pharmaceutical dosage form for administration to a patient to treat bone disease comprising a compacted core containing a therapeutic bis-phosphonate embedded in a gastric retention composition that forms a shell around the core, and a coating of a vitamin D derivative that stimulates transport of calcium from the intestine into the bloodstream applied over the shell, wherein upon contact with gastric fluid the vitamin D derivative is released from the coating, the gastric retention composition swells to promote retention of the dosage form in the patient's stomach for a prolonged period of time, the bis-phosphonate is released at least about an hour after the vitamin D derivative is released and the dosage form degrades into particles too small to cause gastric retention.

20. The oral pharmaceutical dosage form of claim 19 wherein the core slows release of the bis-phosphonate.

21. The oral pharmaceutical dosage form of claim 19 wherein the core has a coating that slows or delays release of the bis-phosphonate.

22. An oral pharmaceutical dosage form for administration to a patient disease comprising a capsule enclosing:
a) a first tablet comprising a compacted core containing a therapeutic bis-phosphonate, and
b) a second tablet containing a vitamin D derivative that stimulates transport of calcium from the intestine into the bloodstream,
wherein upon contact with gastric fluid, the capsule dissolves, the vitamin D derivative is released from the second tablet, the first tablet expands to promote retention of the dosage form in the patient's stomach, the bis-phosphonate is released from the core at least one hour after the vitamin D derivative is released and the first tablet degrades into particles too small to cause gastric retention.

23. A method of treating bone disease in a human patient in need of such treatment by administering to the patient a dosage form of claim 1.

24. The method of claim 23 wherein the bone disease is metastatic bone disease.

25. The method of claim 23 wherein the bone disease is osteoporosis.

26. The method of claim 23 wherein the bone disease is Paget's disease.

27. A method of inhibiting bone resorption in a human patient in need of such treatment by administering to the patient the pharmaceutical dosage form of claim 1.

28. A method of treating hypercalcemia in a human patient in need of such treatment by administering to the patient the pharmaceutical dosage form of claim 1.

29. A method of treating malignancy in bone of a human patient in need of such treatment by administering to the patient the pharmaceutical dosage form of claim 1.

30. An improved combination therapy for treatment of bone disease by repeat administration to a patient of a unit dosage form that releases a calcium transport stimulator in an immediate or uncontrolled manner and, after swelling to a size that prevents passage through the pylorus, and after a delay time period to allow the calcium transport stimulator to deplete the upper GI tract of calcium, releases a therapeutic bis-phosphonate in the stomach.

31. An improved combination therapy for treatment of bone disease of claim 30 wherein the delay time period is an hour or more.

32. An improved combination therapy for treatment of bone disease of claim 31 wherein the delay time period is from about 2 to about 6 hours.

33. An improved combination therapy for treatment of bone disease of claim 30 wherein the bis-phosphonate is released in the stomach in either an immediate or sustained release manner.

34. A combination therapy method for treating bone disease in a patient in need of such treatment by a combination drug regimen comprising the steps of:
administering to such patient a unit pre-dose of a vitamin D derivative and, about 2 to about 6 hours after administration of the pre-dose, administering a unit dose of a bisphosphonate.

35. The method of claim 34 wherein the vitamin D derivative is selected from the group consisting of calcitriol, alphacalcidol, 24,25-dihydroxy vitamin D₃, and calcifediol.

36. The method of claim 35 wherein the vitamin D derivative is calcitriol.

37. The method of claim 34 wherein the the bis-phosphonate is selected from the group consisting of alendronate, resendronate, etidronate, and tiludronate.

38. The pharmaceutical dosage form of claim 37 wherein the bis-phosphonate is alendronic acid or a pharmaceutically acceptable salt or hydrate thereof.
